(19) United States
(12) Patent Application Publication
(10) Pub. No.: US 2017/0202988 A1
(43) Pub. Date: Jul. 20, 2017

(54) MICROBIAL SANITIZING CHAMBER

(71) Applicant: REGINALD W. CLARK, SAN DIEGO, CA (US)

(72) Inventor: REGINALD W. CLARK, SAN DIEGO, CA (US)

(73) Assignee: NOVATRON, INC., SAN DIEGO, CA (US)

(21) Appl. No.: 15/410,705

(22) Filed: Jan. 19, 2017

Related U.S. Application Data
(60) Provisional application No. 62/280,692, filed on Jan. 19, 2016.

(51) Int. Cl. A61L 2/10; A61L 2/26

(52) U.S. Cl. CPC A61L 2/10; A61L 2/26; A61L 2202/11; A61L 2202/121

(57) ABSTRACT

The present disclosure describes a chamber with an interior UV reflective surface, and a light source emitting an ultraviolet light that is used to kill microorganisms on one or more surfaces of an object, thereby sanitizing the object.

### CROSS REFERENCE TO RELATED APPLICATION

This application claims the benefit of U.S. Provisional Application No. 62/280,692, filed Jan. 19, 2016, entitled MICROBIAL SANITIZING CHAMBER, which is herein incorporated by reference in its entirety for all purposes.

### BACKGROUND

Nosocomial infections, also termed Hospital Acquired Infections (“HAI”), have become a major problem for healthcare facilities and their patients. Nosocomial infections acquired in a hospital or other healthcare setting are often caused by organisms that are resistant to antibiotics. A key factor in this problem is the prevalence of microorganisms such as Clostridium difficile (C. diff), methicillin-resistant Staphylococcus aureus (“MRSA”) and vancomycin-resistant Enterococcus faecalis (VRE) that are resistant to antibiotics. Another issue for healthcare workers and patients is the increasing threat posed by highly virulent organisms such as the Ebola virus.

HAIs are the fourth leading cause of death in the U.S. More people die from HAIs in the U.S. than from AIDS, breast cancer and auto accidents combined. In a 2009 report, HAIs were estimated to affect 2 million people annually, resulting in more than 100,000 deaths and costs of $45 billion.

A recent study found that a significantly greater number of microorganisms reside on hand-held objects such as remote control devices, “pillow speaker” devices and other hand-held devices used by healthcare workers and patients in hospital rooms than on other common surfaces such as tray tables, bathroom surfaces, door knobs, hand rails, etc. Also, since small hand-held objects are handled more frequently, they constitute a major pathway for transmitting microorganisms. A device for quickly and effectively disinfecting (or sanitizing) small, hand-held objects frequently used in healthcare environments would provide an important means of addressing HAI problems.

The anti-microbial effects of ultraviolet (UV) light are well known. UV wavelengths have been used in hospitals and in other settings to kill microorganisms since the early 1900's. In general, UV energy causes germicidal effects by interacting with the DNA of microorganisms and creating thymine dimers within the DNA strands. This disruption of the DNA prevents the organism from functioning and reproducing. Effective UV wavelengths for inactivation of microorganisms are typically, from about 220 nm to about 280 nm, with maximum effectiveness near 265 nm.

U.S. Pat. Nos. 7,875,247 and 8,404,186 describe methods and apparatuses for efficiently achieving very high destruction levels (more than 6 orders of magnitude reduction) of resistant microorganisms in flowing air using reflective cavity technology. While such reduction levels of microorganisms are not achievable on surfaces because of surface imperfections that can prevent exposure of organisms to the UV light, with an appropriate exposure configuration and an appropriate UV dose, it is possible to achieve significant reductions in microbial contamination on surfaces. Such reductions of a few orders of magnitude (for example, 1 to 5 orders of magnitude, also described as 1 to 5 logs) are referred to herein as “disinfection” or “sanitization” of a surface.

### SUMMARY

Described herein are methods and apparatuses (chambers) for disinfecting portable objects and devices using ultraviolet (UV) light. The methods and apparatuses (chambers) described herein can also be used to disinfect non-portable objects and devices using UV light. The methods and apparatuses (chambers) of the disclosure can be used to disinfect a surface. In addition, the methods and apparatuses (chambers) of the disclosure can be used to reduce the presence of, eliminate, or prevent the growth of microorganisms, such as pathogenic microorganisms, on a surface.

In one embodiment, an object to be disinfected is placed in an enclosed chamber where it is exposed to UV light of an appropriate intensity for a period of time sufficient to reduce the presence of, eliminate, or prevent the growth of microorganisms on the surface of the object.

Kill of microorganisms by UV depends on the total UV energy (also known as “UV dose”) applied. The UV energy (E) is the product of the incident UV power (also known as “UV irradiance”) and time. UV energy can be measured in, for example, Joules/cm² (J/cm²), and power or irradiance can be measured in units of Watts/cm² (W/cm²).

E (J/cm²)=P (W/cm²)×t (sec) (1)

Several challenges must be addressed to properly disinfect objects with UV light. First, sufficient UV energy must be applied to all parts of the surface of the object to assure that all areas are properly disinfected. Second, some devices such as “pillow speakers” and communication devices used in healthcare settings have cords (for example, electrical cords or tubing) attached that cannot be disconnected from the device. If the object is placed in an enclosed chamber for disinfection, it must be possible to have any electrical cords attached to the object extend outside the chamber during the disinfection process. Third, it is important that personnel operating the disinfection device not be exposed to UV light in order to prevent damage to skin and/or eyes. Thus, it is important that the disinfection device be designed to prevent the escape of UV light into the external environment. As a practical matter, the design of the disinfection device must also be such that medical personnel can easily use it without the requirement for training or expertise in UV technology. In addition, the cost of the disinfection device must be low enough that hospitals and other medical facilities can afford to install or place them in each hospital room or other healthcare location in close proximity to the objects that require disinfection.

In one embodiment of the disinfection apparatus described herein, a disinfection chamber consists of an enclosed chamber with one or more UV lamps, a reflective inner surface, and a port for allowing an electrical cord that may be attached to the object to extend to the exterior of the chamber (unit). The disinfection unit described in this embodiment can be mounted, for example, on a wall near the bed in a hospital room.

One of the most important features of a UV disinfection unit of the type described herein is to provide high UV irradiance to all parts of the object being disinfected. Since the object being disinfected must be supported during the disinfection process, the support structure must not interfere with the transmission of the UV light to all areas of the object. One approach would be to use a metal or plastic grid or grill to support objects to be disinfected. However, the metal or plastic would create areas where the UV light would not reach the object and would lead to areas that would not be disinfected.

Most materials, including plastics are poor transmitters of the UV germicidal wavelengths, for example, near 265 nm, which are effective for microbial disinfection. Some quartz materials, however, have good transmission of UV germicidal wavelengths.

It would be possible to use a quartz plate to support the object. However, as a practical matter, because of manufacturing processes involved in producing quartz plates, they are very expensive. As a result, use of a quartz plate to support the objects to be disinfected would lead to an overall cost for the disinfection unit that would interfere with its adoption, making it not practical for use in individual hospital rooms near the objects to be disinfected.

An embodiment of the apparatus described herein uses an alternate approach to supporting objects to be disinfected that would allow good exposure, and at a reasonable cost. This approach uses an array of quartz rods as the support structure.

In one embodiment of the apparatus, a port as described herein is incorporated near the door of the apparatus to allow the electrical cord attached to a hand-held object to exit the interior UV exposure chamber. This port is designed to minimize exposure of personnel to the UV rays in the interior UV exposure chamber.

The apparatus described herein incorporates simple and inexpensive automated controls to facilitate operation by hospital and other healthcare personnel without the need for special training, while still assuring that objects are adequately treated to greatly diminish the number of microorganisms on an object being disinfected.

Provided herein is a sanitizing chamber, comprising one or more UV reflective interior surfaces, one or more light sources emitting ultraviolet light, and a door on one surface of the chamber. An object can be placed through the door into the interior of the chamber and upon turning on the one or more UV light sources, one or more surfaces of the object are sanitized. In one embodiment, the reflective surface comprises a reflective material having a reflectivity of 50% to 60%, 60% to 70%, 70% to 80%, 80% to 90%, or 90% to 99.999%, or is coated with a reflective material having a reflectivity of 50% to 60%, 60% to 70%, 70% to 80%, 80% to 90%, or 90% to 99.999%. In alternative embodiments, the chamber can further compromise one or more of the following: a support structure for the object being disinfected, an electrical system, an interlock switch, an activation switch, a cycle indicator, a view port indicator, or a port connecting the interior of the chamber with the exterior of the chamber. The port can optionally comprise one or more covers. The sanitizing chamber can be a stand-alone chamber or be mounted on or attached to a wall or other support. The sanitizing chamber can be portable or not portable.

Also provided herein is a sanitizing chamber, comprising: an interior chamber comprising one or more reflective surfaces and one or more light sources emitting ultraviolet light; a door on one surface of the chamber; a support; an electrical system; an interlock switch; an activation switch; a cycle indicator; and a view port indicator. In an alternative embodiment, the sanitizing chamber comprises one or more ports connecting the interior of the chamber to the exterior of the chamber. The sanitizing chamber can be portable or not portable.

An alternative embodiment is a sanitizing chamber, comprising: an exterior chamber and an interior chamber, wherein the interior chamber comprising one or more reflective surfaces and one or more light sources emitting ultraviolet light; and a door on one surface of the exterior chamber. An object can be placed through the door into the interior chamber and upon turning on the one or more light sources, one or more surfaces of the object are sanitized.

Also disclosed herein are methods of sanitizing an object or the surface of an object, comprising: obtaining any one of the sanitizing chambers as described herein, placing the object through the door of the chamber; placing the object onto a surface of the interior chamber or onto a support; closing the door; and turning on the one or more light sources emitting ultraviolet light.

Provided herein are chambers for sanitizing an object or the surface of an object, comprising: a) an interior and an exterior; b) one or more internal UV reflective surfaces comprising a UV reflective material; c) one or more supports to place the object upon; d) one or more light sources that emit ultraviolet light; and e) a door on one surface of the chamber in which the object can enter or exit the chamber. In other embodiments, the chamber further comprises: a) a port connecting the interior of the chamber with the exterior of the chamber; b) an electrical system; c) an interlock switch; e) an activation switch; f) a cycle indicator; or g) a view port indicator. In alternative embodiments, the UV reflective surface comprise a UV reflective material having a reflectivity of 50% to 60%, 60% to 70%, 70% to 80%, 80% to 90%, or 90% to 99.999%, or is coated with a UV reflective material having a UV reflectivity of 50% to 60%, 60% to 70%, 70% to 80%, 80% to 90%, or 90% to 99.999%; b) the reflective material comprises: aluminum; Miro® 4300 UP UV; Polytetrafluoroethylene (PTFE); expanded PTFE (ePTFE), or a combination of aluminum, Miro® 4300 UP UV, Polytetrafluoroethylene (PTFE), or expanded PTFE (ePTFE); c) the reflective material has a UV reflectivity of greater than 80%, or a UV reflectivity of 80% to 81%, 81% to 82%, 82% to 83%, 83% to 84%, 84% to 85%, 85% to 86%, 86% to 87%, 87% to 88%, 88% to 89%, 89% to 90%, 90% to 91%, 91% to 92%, 92% to 93%, 93% to 94%, 94% to 95%, 95% to 96%, 96% to 97%, 97% to 98%, 98% to 99%, or 99% to 100%; or d) the reflective material is a surface coated with a UV reflective paint. In other embodiments, a) the one or more supports comprise quartz; or b) the one or more supports comprise a pocket in which the object can be placed. In alternative embodiments, the emitted ultraviolet light is from about 100 nm to about 300 nm, from about 220 nm to about 280 nm, from about 240 nm to about 280 nm, from about 220 nm to about 320 nm, or from about 250 to 270 nm, or about 254 nm or about 265 nm. Also disclosed herein are methods of sanitizing an object or the surface of an object, comprising: a) obtaining a chamber as described herein; b) placing the object through the door of the chamber; c) placing the object onto the one or more supports; d) closing the door; and e) turning on the one or more light sources emitting ultraviolet light. Also disclosed herein are methods of reducing microbes on a surface of an object, comprising: a) obtaining a chamber as described herein; b) placing the object through the door of the chamber; c) placing the object onto the one or more supports; d) closing the door; e) turning on the one or more light sources emitting ultraviolet light; and f) reducing the microbes on the surface about 1 to about 5 logs magnitude or about 0.5 to about 5 logs magnitude.

Also provided herein are chambers for sanitizing an object or the surface of an object, comprising: a) an interior chamber comprising one or more internal UV reflective surfaces comprising a UV reflective material; b) an outer chamber comprising an exterior surface; c) one or more supports to place the object upon; d) one or more light sources that emit ultraviolet light; and e) a door on one surface of the chamber in which the object can enter or exit the chamber. In alternative embodiments, the chamber further comprises: a) a port connecting the exterior surface of the outer chamber with the internal UV reflective surfaces of the interior chamber; b) an electrical system; c) an interlock switch; e) an activation switch; f) a cycle indicator; or g) a view port indicator. In alternative embodiments, the UV reflective surface comprise a UV reflective material having a reflectivity of 50% to 60%, 60% to 70%, 70% to 80%, 80% to 90%, or 90% to 99.999%, or is coated with a UV reflective material having a UV reflectivity of 50% to 60%, 60% to 70%, 70% to 80%, 80% to 90%, or 90% to 99.999%; b) the reflective material comprises: aluminum; Miro® 4300 UP UV; Polytetrafluoroethylene (PTFE); expanded PTFE (ePTFE), or a combination of aluminum, Miro® 4300 UP UV, Polytetrafluoroethylene (PTFE), or expanded PTFE (ePTFE); c) the reflective material has a UV reflectivity of greater than 80%, or a UV reflectivity of 80% to 81%, 81% to 82%, 82% to 83%, 83% to 84%, 84% to 85%, 85% to 86%, 86% to 87%, 87% to 88%, 88% to 89%, 89% to 90%, 90% to 91%, 91% to 92%, 92% to 93%, 93% to 94%, 94% to 95%, 95% to 96%, 96% to 97%, 97% to 98%, 98% to 99%, or 99% to 100%; or d) the reflective material is a surface coated with a UV reflective paint. In other embodiments, a) the one or more supports comprise quartz; or b) the one or more supports comprise a pocket in which the object can be placed. In alternative embodiments, the emitted ultraviolet light is from about 100 nm to about 300 nm, from about 220 nm to about 280 nm, from about 240 nm to about 280 nm, from about 220 nm to about 320 nm, or from about 250 to 270 nm, or about 254 nm or about 265 nm. Also disclosed herein are methods of sanitizing an object or the surface of an object, comprising: a) obtaining a chamber as described herein; b) placing the object through the door of the chamber; c) placing the object onto the one or more supports; d) closing the door; and e) turning on the one or more light sources emitting ultraviolet light. Also disclosed herein are methods of reducing microbes on a surface of an object, comprising: a) obtaining a chamber as described herein; b) placing the object through the door of the chamber; c) placing the object onto the one or more supports; d) closing the door; e) turning on the one or more light sources emitting ultraviolet light; and f) reducing the microbes on the surface about 1 to about 5 logs magnitude or about 0.5 to about 5 logs magnitude.

### BRIEF DESCRIPTION OF THE DRAWINGS

These and other features, aspects, and advantages of the present disclosure will become better understood with regard to the following description, appended claims and accompanying figures where:

FIG. 1 is a three dimensional isometric perspective drawing of an embodiment of the UV disinfection apparatus described herein for disinfecting objects.

FIG. 2 is a plot of the transmission of several types of quartz as a function of wavelength.

FIG. 3 is a cross-sectional sketch of the interior of an embodiment of the UV disinfection apparatus described herein. The cross-sectional cut is made at the mid-point of the apparatus in the direction perpendicular to the longitudinal axes of UV lamps and support quartz rods used in the apparatus.

FIG. 4 is a plot of the UV reflectivity of Alanod Miro® 4300 UP UV reflective material.

FIG. 5 is a cross-sectional sketch of the interior of an embodiment of the UV disinfection apparatus described herein, where the cross-sectional cut is made at the mid-point of the apparatus in the direction parallel to the longitudinal axes of the UV lamps and the quartz rods.

FIG. 6A, FIG. 6B, and FIG. 6C are drawings showing the incorporation of a port in the apparatus for an electrical cord attached to an object to be disinfected. FIG. 6A is an isometric drawing showing the electric cord port and two optional port covers on the left side of the unit. FIG. 6B is a side view of the unit showing where the cord enters the port. FIG. 6C is an isometric drawing showing an optional sliding cover for the port. FIG. 6D is an isometric drawing showing the electric cord port on the left side of the unit. FIG. 6E is a close up view of the unit in FIG. 6D showing the area where the cord enters the port.

FIG. 7 is a plot of published information on nucleic acid UV absorption and antimicrobial effects as a function of wavelength for two common microorganisms: Escherichia coli (left), and Staphylococcus aureus (right). The broken lines show absorption and the solid lines show the action spectrum for kill of the two microorganisms. (Gates, F. L., 1930, J. Physiol. 14: 31-42.)

FIG. 8 shows published data for UV destruction of three common pathogenic HAI organisms: Clostridium difficile (C. diff.), Methicillin-resistant Staphylococcus aureus (MRSA), and vancomycin-resistant Enterococcus faecalis (VRE). Data courtesy of BioMed Central Ltd from a study by M. Nerandzic, J. Cadnum, M. Pultz and C. Donskey, published in BMC infectious Diseases, 2010, 10; 197.

FIG. 9 shows published data for UV destruction of three exemplary viruses, Vaccinia virus, Lassa virus, and Ebola virus. (J. Sagripanti and D. Lytle, 2011, Arch. Virol. 156: 489-494.)

FIG. 10 shows an isometric drawing showing the location of test points for UV irradiance measurements in the interior UV sanitizer reflective chamber.

FIG. 11 shows data for destruction of MRSA, obtained using an apparatus of the type described herein.

FIG. 12 shows data for destruction of C. difficile, obtained using an apparatus of the type described herein. Data from two test replications are shown.

FIG. 13 shows a way of assuring that an object placed in the sanitizer unit is in a central location. FIG. 13 shows a “pocket” configuration that makes sure the object isn't placed against, for example, one of the front-back walls where it wouldn't receive any reflected UV rays on the sides. In this embodiment, several of the quartz support rods are lowered to provide an area where the object (for example, an IPad®) can be placed, leaving room on the side for reflected UV rays from the side walls of the cavity to reach the sides of the object being disinfected.

### DETAILED DESCRIPTION

The following detailed description is provided to aid those skilled in the art in practicing the present disclosure. Even so, this detailed description should not be construed to unduly limit the present disclosure as modifications and variations in the embodiments discussed herein can be made by those of ordinary skill in the art without departing from the spirit or scope of the present inventive discovery.

Embodiments of the disclosure will now be described with reference to the accompanying Figures, wherein like numerals refer to like elements throughout. The terminology used in the description presented herein is not intended to be interpreted in any limited or restrictive manner, simply because it is being utilized in conjunction with a detailed description of certain specific embodiments of the disclosure. Furthermore, embodiments of the disclosure may include several novel features, no single one of which is solely responsible for its desirable attributes or which is essential to practicing the disclosure herein described.

As used in this disclosure and the appended claims, the singular forms “a”, “an” and “the” include a plural reference unless the context clearly dictates otherwise. As used in this disclosure and the appended claims, the term “or” can be singular or inclusive. For example, A or B, can be A and B.

### OBJECTS TO BE DISINFECTED

An object of the disclosure can be a hand-held object or an object that is not a hand-held object. The object can be a device. The object can be, for example, a hand held remote control for a television or other apparatus, a “pillow speaker”, a tray, a holder, a medical instrument, or a medical device. The object can be anything that is found in a medical facility, including, for example, a doctor's office, medical office, or hospital. An object can have a cord, or an extension, or tubing that is attached to it. An object can also be without a cord, or an extension, or tubing attached to it.

A medical device is an instrument, apparatus, implement, machine, contrivance, implant, or other similar or related article, including a component part, or accessory, which is intended for use in the diagnosis of disease or other conditions, or in the cure, mitigation, treatment, or prevention of disease, in man or other animals. The medical device can be used externally (outside the body) or internally (inside the body).

An object can be an electronic device. Exemplary electronic devices are: an Apple® IPad®, a laptop computer, an Apple® IPhone®, any Smart phone, a keyboard, or any communication device or computer device that can fit inside any of the chambers disclosed herein. An object can also be a device, instrument, tool, or utensil. For example, any utensil used in food preparation or in a restaurant can be disinfected using any of the disclosed chambers.

In addition to uses in medical and healthcare environments, disinfection devices of the type described herein can be used in a number of other applications where disinfection of objects is desirable. For example, disinfection units of the type described herein can be used in restaurant and other food service environments to sanitize small hand held electronic devices such a Apple® Wad® used to record food orders, as well as food preparation utensils. Another application of the device is in schools where objects frequently handled by students and teachers can be sanitized to prevent the transfer of contagious illnesses.

It will be apparent to one skilled in the art that many applications exist for the device in addition to the medical and healthcare applications described herein. Although detailed descriptions are provided herein regarding medical and healthcare applications, nothing herein should be interpreted to limit the use of the device described herein to medical and healthcare applications.

The object can be placed in any configuration inside the chamber. For example, the object can be “standing up” or on its side. The object can be removed after a treatment (UV dose) and repositioned in a different configuration for a second treatment. Multiple treatments and/or configurations can be conducted prior to use of the object.

Sanitizing

The term sanitizing can be used interchangeably throughout the disclosure with the term “disinfecting”. Sanitizing is a reduction in the presence of microorganisms, the elimination of the presence of microorganism, or a reduction in the growth of microorganisms.

The number and type of microorganisms an individual encounters and the health and functionality of the immune system of the individual determines the likelihood that the individual will acquire an infection. If the number of microorganisms is reduced to a level that is low enough that no infection will occur, the level is said to be below the “infectious dose” for the individual and the organism in question. The goal of sanitization of the type described herein is to reduce the microorganism populations on common objects used in healthcare and other public environments to levels that are below the infectious dose for pathogenic microorganisms for most individuals.

Sanitizing can be, for example, a reduction of the presence of viable microbes by 1 to 5 logs, which equates to a reduction of 10 to 100,000 times. For example if an object has 1,000 pathogens per cm², a 4 log reduction would result in an average microorganism count of 0.1 pathogens per cm², or an average density of one microorganism every 10 cm². Such a low population would be far below the level that would be expected to cause an infectious dose for almost any pathogen in almost any individual.

Chamber

The term “chamber” can be used interchangeably throughout the disclosure with the terms “apparatus” and “unit.” The chamber has an internal (interior) chamber where the object is placed.

The chamber can be hand-held or not hand-held. The shape and dimensions will be determined by one skilled in the art and will depend on the object(s) to be sanitized. The chamber can be, for example, a rectangle, a square, or a closed cylinder.

For example, if the chamber is a rectangle, the width can be from 5 to 50 inches or from 12 to 30 inches, the height can be from 5 to 12 inches or from 4 to 24 inches, and the depth can be from 4 to 10 inches or from 3 to 30 inches.

The apparatus may comprise an outer chamber that surrounds an inner (interior) chamber. The outer chamber can be made of metal, plastic or any other suitable material.

The chamber can weigh, for example, 2 to 15 pounds, or 4 to 11 pounds, or 8 to 100 lbs.

The internal (interior) chamber will have dimensions that will fit inside the outer chamber. The internal chamber can be fabricated from, lined with, or painted (or coated) with a reflective material, or both.

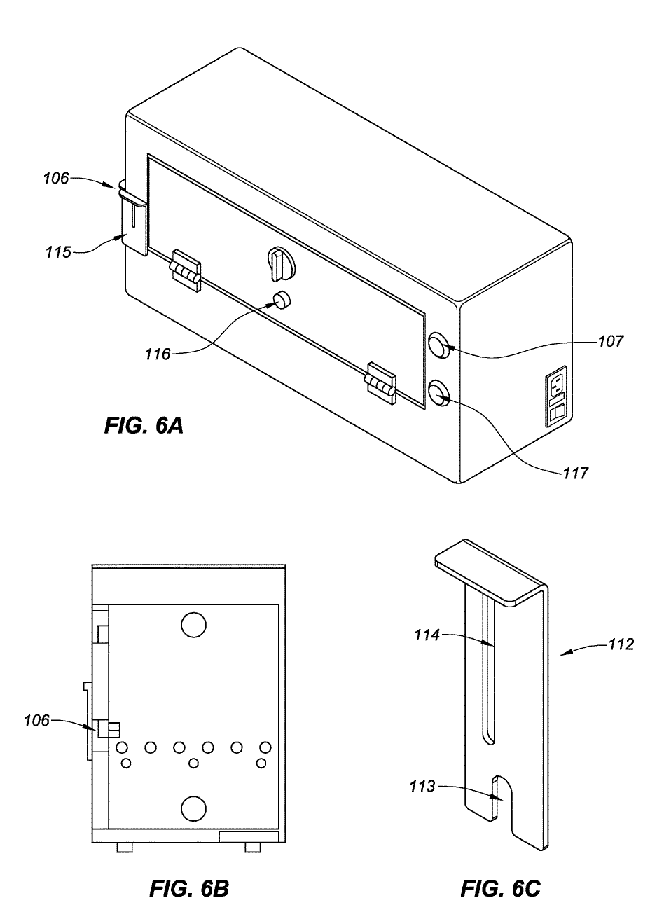

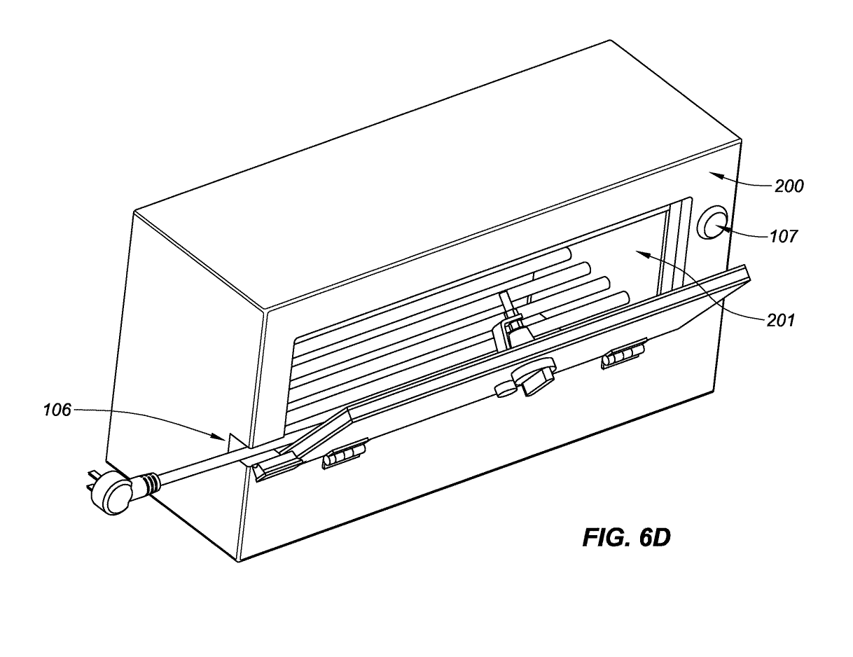

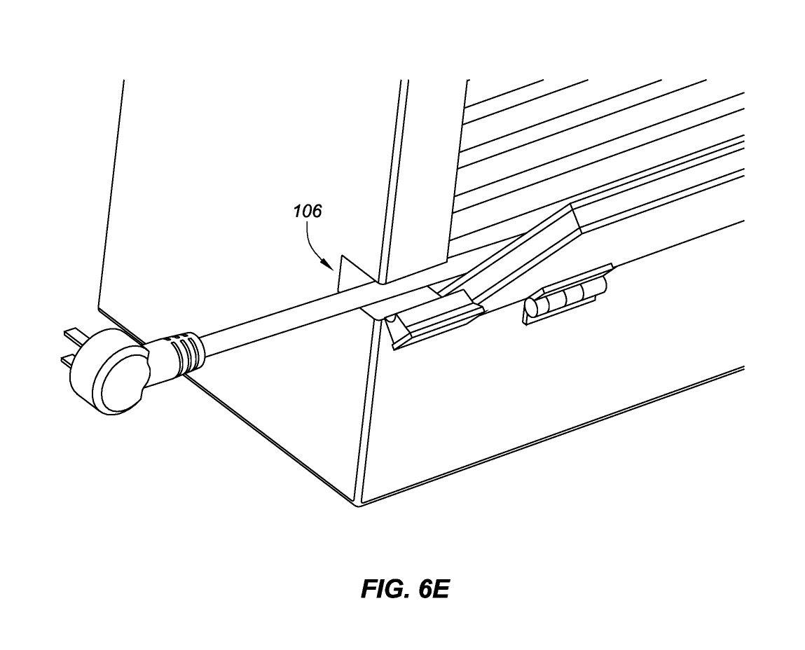

In one embodiment the apparatus (chamber) can comprise an interior chamber and an outer chamber. An exemplary outer chamber 200 and an exemplary interior chamber 201 are shown in FIG. 6D. In another embodiment, the apparatus may consist of a single interior chamber. The single interior chamber may advantageously be fabricated from or lined on its interior surface with a UV reflective material Electrical, electronic and other components may be attached to the exterior of the single interior chamber and enclosed in covered spaces as may be desirable.

The chamber can have a door that can be opened, placed on one side or surface of the chamber. In addition, the exterior of the chamber will have a view port so that the user can verify that the UV lamps are on in the interior chamber. In addition, the chamber will have an activation (on/off) switch, and may have a cycle indicator to ensure that each cycle that is run is complete.

The chamber can have an optional port in which a cord, extension, or tubing that is connected to an object can be placed through. For example, an electrical cord attached to an object can be passed through the port from the interior of the chamber to the outside (or exterior) of the chamber.

Power Supply

In one embodiment, the UV lamps and electronic controls, described herein, may be powered from conventional alternating current (AV) electrical “wall plug” electrical power of the type normally available in residential, commercial, industrial and healthcare settings (for example, 120 volt AC wall plug electrical power).

In another embodiment, the electrical power for the disinfection unit can be provided by a rechargeable battery pack that is combined with a battery charging electrical power supply and an inverter to convert the low voltage dc battery power to AC power of the appropriate voltage and power level to energize the UV lamps and operate the electronic controls.

Reflective Surfaces

The interior surface of the chamber can be fabricated from, or painted (or coated), or lined with, a UV reflective material on its interior surface.

The UV reflective material can be composed of aluminum, which has moderately good reflectivity in the UV, or from a material with significantly higher reflectivity in the UV such as Miro® 4300 UP UV reflective material manufactured by Alanod Gmbh & Co. in Germany, or some other material coated with a UV reflective paint such as the UV-Max paint manufactured by Lumacept, Inc.

The reflective material may consist of Polytetrafluoroethylene (PTFE) or expanded PTFE (ePTFE), or other similar materials which have high UV reflectivity. In one embodiment, the reflective material may consist of one or more of Spectralon, which has a UV reflectivity of about 94%, ODM, manufactured by Gigahertz-optic, which has a UV reflectivity of about 95%, or DRP, available from W. L. Gore and Associates, which has a UV reflectivity of about 99.4% to 99.9%. These materials have diffuse or Lambertian reflective characteristics. Such diffuse reflective surfaces reflect incident UV rays that strike the surface at a single angle with respect to the normal to the surface into a variety of angles with respect to the direction normal to the surface. Reflective surfaces with diffuse reflective characteristics can advantageously improve the uniformity of UV irradiance in a reflective chamber.

Other UV reflective coatings could constructed from a mixture of a binder and reflecting additives such as barium sulfate, magnesium fluoride, magnesium oxide, aluminum oxide, holmium oxide, calcium oxide, lanthanum oxide, germanium oxide, tellurium oxide, europium oxide, erbium oxide, neodymium oxide, samarium oxide, or ytterbium oxide.

If the chamber is a rectangle or square, then it may be desirable that all the six interior surfaces be reflective. If the chamber is cylindrical, then the interior surface of the cylinder will be reflective along with the two ends of the cylinder. As described above, the chamber can be any shape that is practical to fabricate. For example, a chamber can comprise three or more sides.

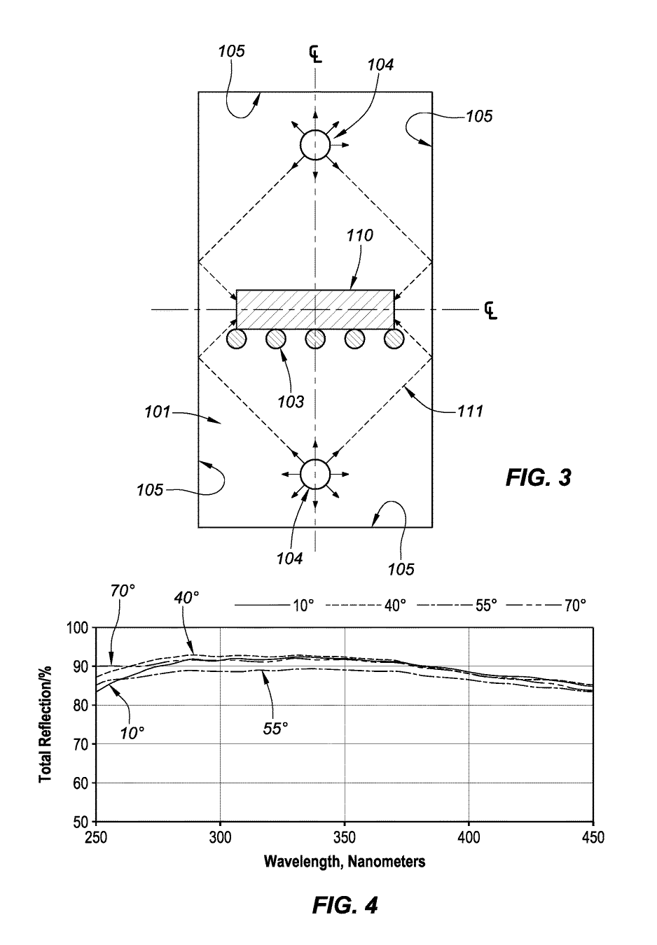

An advantage of having all the interior surfaces of the treatment chamber fabricated from, or lined with reflective material is that, as depicted in FIG. 3, reflected UV rays can reach areas on the object being sanitized that would not otherwise be exposed to the sanitizing UV rays.

A Support

A support for holding an object can be made of a metal, plastic, wire, or quartz, or a combination of any one or more of the above. A support can be, for example, a solid structure, a flexible structure, a flat structure, a plate, a grid, a grill, a patterned structure, a mesh, a wire mesh, or any configuration that can support an object. A support can be one or more rods, for example, an array of rods. Exemplary cylindrical rods are shown in FIG. 6D.

A support can be made of any material that has good transmission of UV germicidal wavelengths, for example a wavelength of from about 220 nm to about 300 nm. The shape of a “support” can be cylindrical, triangular, square, or any shape that allows for an object to be placed upon the support.

A support can be one or more rods. One or more rods can be placed in or attached to the interior chamber. A rod can be hollow or solid. A rod can be made of a quartz material. Exemplary quartz materials are commercial grade fused quartz, or optical grade fused quartz. Quartz is semi-transparent to UV germicidal wavelengths and is a suitable material for transmitting UV light. A support can also be made of fused silica. Rods can be of any diameter that has sufficient strength and rigidity to support objects that will be placed on them. The number of rods used and the horizontal spacing between the rods will be such that the objects they will support will not fall between them. For example, a support can comprise two or more rods, or 2 to 20 rods.

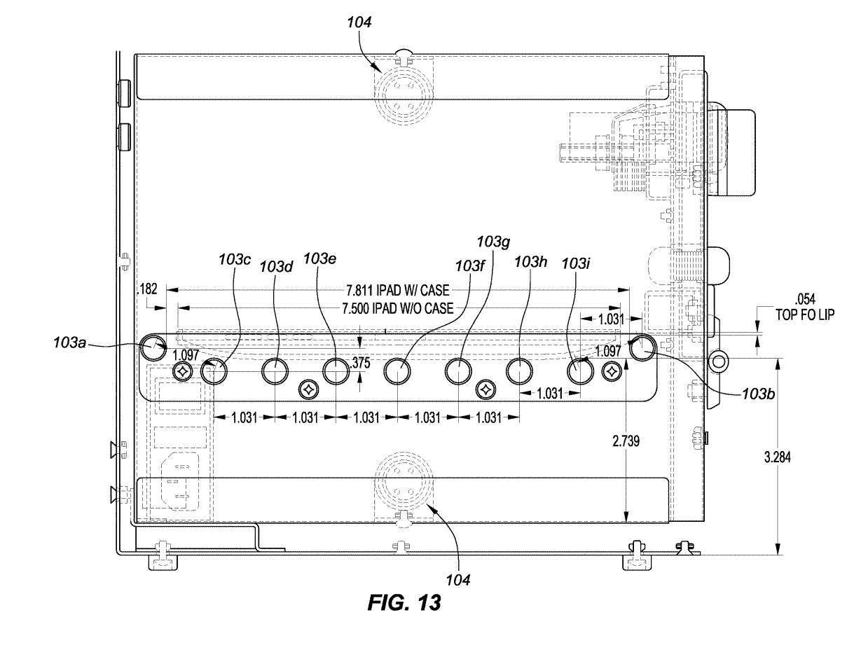

One or more rods can form a support that is flat but also comprises a “pocket”, see FIG. 13. FIG. 13 shows a way of assuring that an object placed in the sanitizer unit is in a central location. FIG. 13 shows a “pocket” configuration that makes sure the object isn't placed against, for example, one of the front-back walls where it wouldn't receive any reflected UV rays on the sides. In this embodiment, several of the quartz support rods (103c to 103i) are lowered to provide an area where the object (for example, an IPad®) can be placed, leaving room on the side for reflected UV rays from the side walls of the cavity to reach the sides of the object being disinfected. In FIG. 13 two rods 103a and 103b are in a different plane than rods 103c to 103i. Two or more rods (for example, 103a and 103b) should be on a different plane than the remaining rods (for example 103c to 103i), and each of the two or more rods should be on opposite sides of the remaining rods, as shown in FIG. 13. An alternative “pocket” formation can be, for example, a mesh that has a lower portion into which an item is placed, such as an IPad®.

Ultraviolet Light

Ultraviolet light is part of the electromagnetic spectrum, which is classified into three wavelength ranges: UV-C, from 100 nanometers (nm) to 280 nm; UV-B, from 280 nm to 315 nm; and UV-A, from 315 nm to 400 nm.

UV-C light is germicidal, it deactivates the DNA of bacteria, viruses and other pathogens and thus destroys their ability to multiply and cause disease. Specifically, UV-C light causes damage to the nucleic acid of microorganisms by forming covalent bonds between certain adjacent bases in the DNA. The formation of such bonds prevents DNA from being unzipped for replication, and the organism is unable to reproduce. When the organism tries to replicate, it dies.

The source of UV-C light is from one or more lamps or other UVC sources. Exemplary lamps and UV sources that can be used in the internal chamber of the apparatus are low-pressure mercury germicidal lamps, medium-pressure mercury lamps and UV LED sources. Exemplary distributors of germicidal lamps are Light Sources, Inc. and Heraeus-Noblelight Fusion UV, Inc.

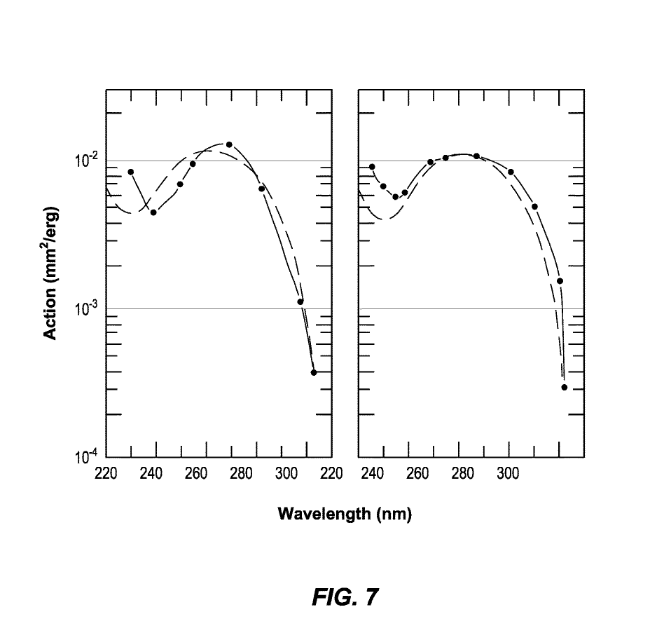

Exemplary UV wavelengths that can be used in apparatuses described herein are: from about 100 nm to about 300 nm, from about 220 nm to about 280 nm, from about 240 nm to about 280 nm, from about 220 nm to about 320 nm, or from about 250 to 270 nm, or about 254 nm or about 265 nm. The UV dose used in the internal chamber is sufficient to cause germicidal effects by interacting with the DNA of microorganisms and creating thymine dimers within the DNA strands causing the exposed microorganisms to be unable to function and reproduce. For example, low pressure mercury UV germicidal lamps produce copious amounts of UV radiation from the strong mercury atomic radiation line centered at 253.7 nm, which is in the wavelength region between 220 nm and 300 nm where a broad peak occurs in germicidal effects as shown in FIG. 7.

UV Exposure Time

The exposure time necessary to provide a desired UV dose with a known UV irradiance can be determined by one of skill in the art in accordance with Equation 1 herein. As the UV intensity increases, the time required to treat an object to a desired dose level decreases. Exemplary doses and times are greater than 50 mW-s/cm² provided in an exposure time of 60 seconds or less. Other exemplary doses are 10 mW-s/cm² to 500 mW-s/cm². Other exemplary exposure times are less than 5 minutes.

Short-wave UV light is harmful to humans. In addition to causing sunburn and (over time) skin cancer, this light can produce extremely painful inflammation of the cornea of the eye, which may lead to temporary or permanent vision impairment. It can also damage the retina of the eye. For this reason, the light produced by a germicidal lamp must be carefully shielded against both direct viewing and reflections and dispersed light that might be viewed. As described herein, the chamber of the disclosure comprises a port (as shown in, for example, in FIG. 6A, FIG. 6B, FIG. 6D, and FIG. 6E) that permits electrical cords and/or tubes attached to an object to be sanitized to exit the interior chamber, but helps to protect user from the harmful short-wave UV light.

Integrating sphere optics equations can be used to approximate the photon flux within a reflective chamber. While derived for a spherical geometry, the results are based on an infinite power series of multiple reflections and give a reasonable approximation of a non-spherical geometry as long as the overall dimensions (length, width, etc.) are approximately equal.

The irradiance on the inside surface of an integrating sphere is given by the equation:

$${P}_{s} = \frac{{P}_{in}}{{A}_{s}} \times \frac{R}{1 - R ( 1 - α )} = {P}_{in} \frac{M}{{A}_{s}} , \qquad ( 2 )$$

where Ps is the irradiance or flux density in W/cm² near the inner surface of the sphere, R is the reflectivity of the walls, is the total internal surface area and is the fractional open or absorbing area of the surface. The “multiplier”, M, is a figure of merit given by:

$$M = \frac{R}{1 - R ( 1 - α )} . \qquad ( 3 )$$

This term represents an increase in irradiance due to multiple reflections. For example, the multiplier can be as large as 50 when R=0.99 and the value of a is about 0.01.

Pathogenic Microorganisms.

Pathogenic microorganisms cause disease. Pathogens include, for example, bacteria, viruses, or fungi. A pathogen can be, for example, a pathogenic bacteria or a virus. One skilled in the art could identify the presence of any pathogen that is not listed herein, or not yet evolved, and determine whether the chambers of the disclosure are effective against it. One skilled in the art will understand that different pathogenic microorganisms may require different UV doses to produce a given desired kill level.

Described below are numerous exemplary pathogenic bacteria whose numbers or population can be decreased by the chamber and methods of using the chamber, as described herein. Any of these microorganisms can be present on a surface of an object. This list is non-exhaustive. One skilled in the art could identify the presence of any pathogenic bacteria that is not listed herein, or not yet evolved, and determine whether the compounds of the disclosure are effective against it. New resistant strains evolve frequently.

Pathogen resistance to antibiotics continues to increase and new resistant strains are also increasing. Pathogenic Bacteria can include multidrug-resistant organisms (MDROs). Pathogenic bacteria can be for example, Pseudomonas aeruginosa, Clostridium difficile (C. Diff), Neisseria gonorrhaeae, Carbapenem-resistant Enterobacteriaceae (CRE), Drug-resistant Streptococcus pneumoniae, Drug-resistant Campylobacateria, Drug-resistant non-typhoidal Salmonella, Methicillin-resistant Staphylococcus aureus (MRSA), Drug-resistant Shigella, Extended-spectrum Enterobacteriaceae (ESBL), Vancomycin-resistant Enterococcus (VRE), Multidrug-resistant Acinetobacteria, Multidrug-resistant Pseudomonas aeruginosa, Drug-resistant Salmonella serotype Typhi, Fluconazole-resistant Candida, Drug-resistant Tuberculosis, Clindamycin-resistant Group B Streptococcus, Erythromycin-resistant Group A Streptococcus, or Vancomycin-resistant Staphylococcus aureus.

Methicillin-resistant Staphylococcus aureus (MRSA) is a type of staphylococcus or “staph” bacterium that is resistant to many antibiotics. In medical facilities, MRSA causes life-threatening bloodstream infections, pneumonia and surgical site infections. MRSA is different from other types of staph because it cannot be treated with certain antibiotics such as methicillin.

Vancomycin-resistant enterococci (VRE) are a type of bacteria called enterococci that have developed resistance to many antibiotics, especially vancomycin. Enterococci bacteria live in the intestines and on the skin of a mammal, often without causing problems. But if they become resistant to antibiotics, they can cause serious infections, especially in mammals that are ill or weak. These infections can occur anywhere in the body. Some common sites include the intestines, the urinary tract, and wounds.

Vancomycin-intermediate Staphylococcus aureus (also called VISA) and Vancomycin-resistant Staphylococcus aureus (also called VRSA) are specific types of antimicrobial-resistant bacteria. Persons who develop this type of staph infection may have underlying health conditions (such as diabetes and kidney disease), tubes going into their bodies (such as catheters), previous infections with methicillin-resistant Staphylococcus aureus (MRSA), and recent exposure to vancomycin and other antimicrobial agents.

CRE, which is an abbreviation for carbapenem-resistant Enterobacteriaceae, are a family of bacteria that are difficult to treat because they have high levels of resistance to antibiotics. CRE kills 50% of all patients who are infected. Klebsiella species and Escherichia coli (E. coli) are two examples of Enterobacteriaceae, a normal part of the human gut bacteria, which can become carbapenem-resistant. Types of CRE are sometimes known as KPC (Klebsiella pneumoniae carbapenemase) and NDM (New Delhi Metallo-beta-lactamase). KPC and NDM are enzymes that break down carbapenems and make them ineffective. CRE have become resistant to nearly all the available antibiotics. Almost half of all hospital patients who get bloodstream infections from CRE bacteria die from the infection.

### DETAILED DESCRIPTION OF THE FIGURES

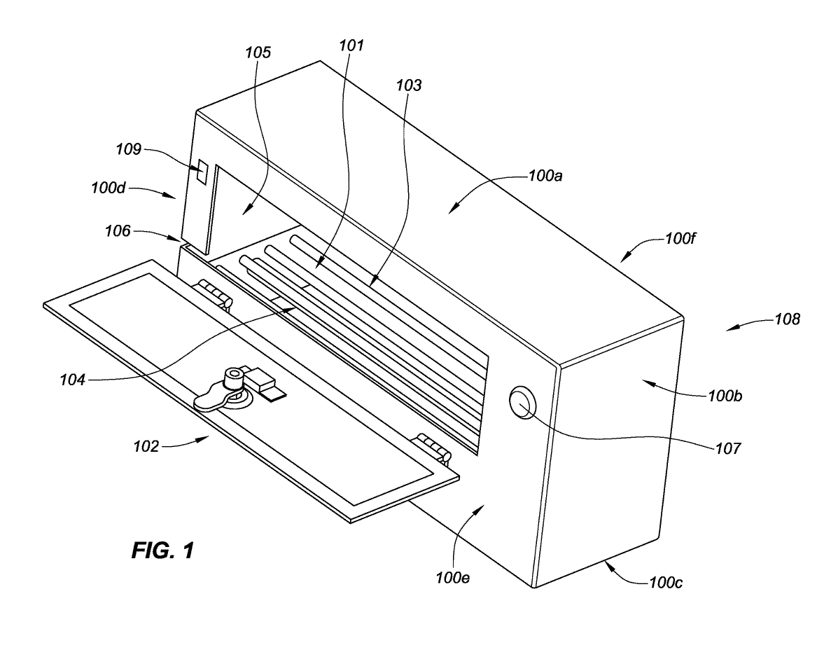

FIG. 1 is an isometric drawing of an embodiment of an apparatus described herein. The apparatus consists of an outer external shell structure (or exterior) (consisting of six sides 100a to 100e) that encloses an interior (UV exposure) chamber 101. The drawing shows a door 102 in the outer external shell structure through which an object can be introduced into the interior (UV exposure) chamber 101 for disinfection. The drawing also shows an array of quartz rods 103 for supporting the object and a UV lamp 104 located near the bottom of the internal UV exposure chamber. Another UV lamp located near the top of the UV exposure chamber is not visible in this view. The device is designed to be lightweight so that it can be suspended, for example, from the support structures attached to the walls near the beds in a hospital or other healthcare facility. The interior UV exposure chamber 101 has an inner reflective surface 105 on each of the interior walls that is designed to reflect UV rays, directing the rays to the exterior surfaces of the object to be disinfected.

An optional port 106 is provided near the door 102 to the unit to allow an electrical cord attached to an object to extend outside the internal UV exposure chamber 101. A gasket material is used to seal the door 102 to the exterior of the unit to help prevent UV light from escaping from the UV exposure chamber 101 into the external environment. One of skill in the art can determine the appropriate gasket material. The door 102 also incorporates an interlock switch 109 to deactivate the UV lamps 104 if the door is opened. The disinfection process is initiated by pressing an activation (on/off) switch 107 shown on the front exterior surface of the unit near the door. The electrical power circuitry for the UV lamps and the electrical circuits and components for timing and control of the disinfection process 108 are located behind the front exterior surface of the unit near the activation switch. The electrical power circuitry and/or the electrical circuits and components (collectively called “the electrical system” 108) can be placed at various locations within the apparatus. A simple witness view port (not shown) allows ordinary light to pass, but blocks harmful UV light, and provides an indication that the UV lamps are functioning. Each lamp 104 is connected in series, so that if one lamp malfunctions, the other lamp(s) will not function. This arrangement provides a very economical method of assuring that the unit is operating properly.

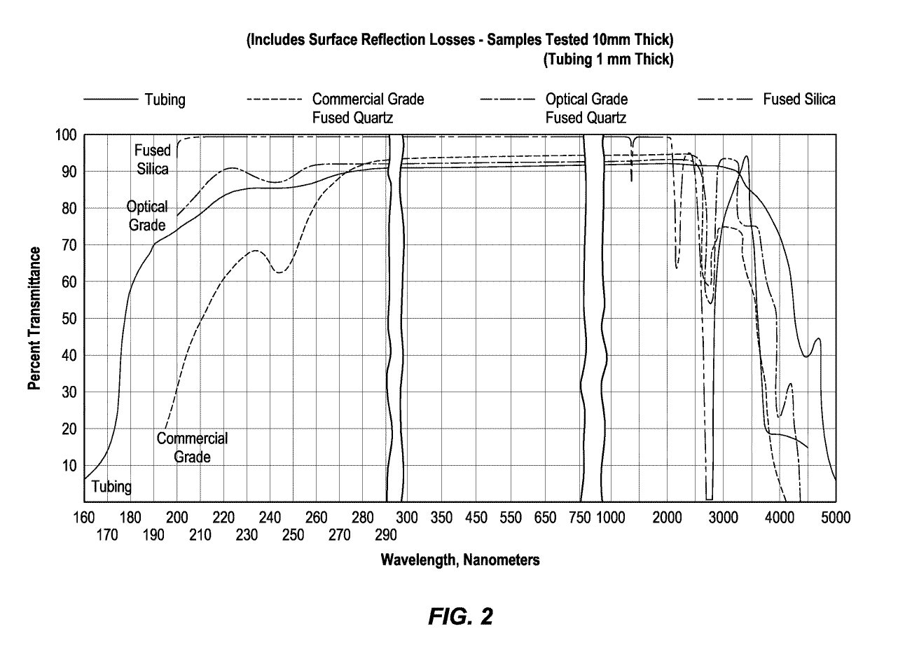

An important requirement for effective UV disinfection of healthcare objects is to assure that the UV light reaches all or most surfaces of the object. To achieve this, a way to support the object (for example an array of rods as shown in FIG. 1 by 103) during UV exposure is needed that will not significantly interfere with UV exposure of the surfaces of the object. Even thin pieces of most materials absorb the UV germicidal wavelengths near 265 nm that are needed for effective microbial disinfection of surfaces. Quartz, however, is an exception. FIG. 2 shows UV transmission curves for several different types of quartz, for example, commercial grade fused quartz and optical grade fused quartz. As can be seen from FIG. 2, transmission near 265 nm (from about 250 nm to about 270 nm), including reflection losses, varies from approximately 80% to near 99% for 10 mm samples of the various types of quartz shown. Thus, quartz is a good material for supporting objects to be disinfected. However, quartz plates are very expensive, making their use impractical for a reasonably priced apparatus designed for installation near a patient's beds in a hospital or other healthcare setting, where there could be one or more units in the hospital or healthcare room. Quartz rods, on the other hand, are relatively inexpensive and, as a result, provide a viable solution for supporting objects to be disinfected in such healthcare settings. It should be noted that quartz plates can be used in any of the apparatuses described herein.

FIG. 3 shows a cross-sectional sketch of the interior chamber 101 of the UV disinfection apparatus. The cross-sectional cut is made perpendicular to the longitudinal axes of the UV lamps 104 and quartz support rods 103. The sketch shows the internal UV exposure chamber 101, the interior reflective surfaces of the exposure chamber 105, two UV lamps 104, one at the top of the apparatus and one at the bottom of the apparatus, several supporting quartz rods 103, an object being exposed 110, and a depiction of UV rays 111 (dotted lines) showing their emission pattern and how they are reflected by the reflective interior surface 105 of the UV exposure chamber to surfaces of the object not exposed to direct rays from the lamps. The object 110 can be placed within the internal chamber 101 in any configuration. In addition, the object 110 can be treated by UV radiation one or more times and/or in one or more different configurations.

There are numerous approaches for creating the reflective interior surface of the UV exposure chamber. One of skill in the art could determine the most appropriate method(s) and material(s). One approach for creating the reflective interior surface of the UV exposure chamber is to fabricate the interior chamber itself from a material that has high UV reflectivity. FIG. 4 shows total reflection data from 250 nm to 450 nm at four different angles of incidence (10, 40, 55, and 70 degrees) for one such commercially available material, Miro® 4300 UP, manufactured by Alanod Aluminium-Veredlung Gmbh & Co. KG in Germany. From the figure, it can be seen that the total reflection in the 250 nm to 260 nm range is between about 85% and 90%. This material is aluminum that has been treated with a special process to create high UV reflectivity.

Another approach is to apply a coating (for example, by painting or spraying) to the inner surface of the UV chamber to enhance its reflectivity at UV germicidal wavelengths. One material that can be applied to increase reflectivity at UV wavelengths is a product developed by Lumacept, Inc. under the product name UVC-Max. This product, when painted onto a surface, increases the UV reflectivity in the 250 nm to 300 nm wavelength range to approximately 60%-75%.

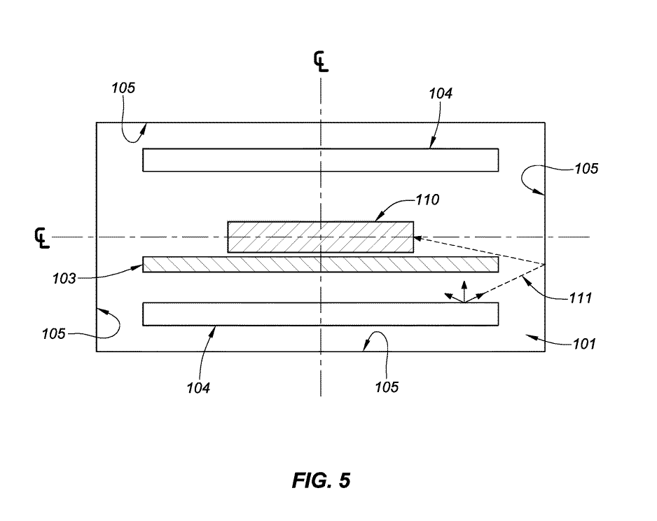

FIG. 5 is a cross-sectional sketch of the interior of the reflective UV exposure chamber 101 with the cross-sectional cut in the direction parallel to the longitudinal axes of the UV lamps 104 and the quartz support rods 103 and passing through the center of the interior exposure chamber 101. The drawing shows the reflective chamber 101, the reflective inner surface of the chamber 105, two UV lamps 104, a cross section of one of the quartz rods 103, a cross section of an object 110 being exposed to UV, and a ray 111 (dotted lines) from one of the UV lamps.

FIG. 6 shows drawings describing a port 106 for an electrical cord attached to an object that is to be disinfected. The port design is to allow electric cords, tubing, or extensions of the object permanently attached to the object to pass through (enter/exit) the sanitizing chamber while preventing the escape of UV light from the device. FIG. 6A is an isometric drawing of one embodiment of the sanitizing device described herein, showing the location of an entrance/exit port 106 for an electric cord on the left side of the unit. FIG. 6B shows a close up view of the entrance/exit port 106 (cord entry). FIG. 6C shows an optional sliding cover 112 that may be used to prevent UV light from escaping from the sanitizing chamber. The sanitizing device may employ one or more sliding covers. The first of the sliding covers 112 may be located on the side of the unit and incorporates a U-shaped slot 113 that closes around the electric cord as well as a vertical slot 114 to allow the sliding cover to move up or down to the correct position to prevent the escape of UV light. The second sliding cover 115 may be located on the front of the unit (FIG. 6A) to prevent the escape of light from the front of the unit. The port 106 incorporates a compressible plastic foam material that is resistant to UV radiation and which seals against the electric cord of the device being disinfected to prevent UV radiation from escaping from the treatment chamber into the external environment where it could cause damage to the operator or other personnel in the vicinity of the disinfection unit. The port 106 also incorporates a curved metal piece that seals against the outer side of the electrical cord and applies pressure to force the electrical cord against the compressible plastic foam material. The port 106 can be located on any of the external walls (100a to 100f) of the apparatus. The port must connect an external wall or walls of the apparatus with the interior chamber of the apparatus. One or more ports can be used in the apparatus. In FIG. 6A a witness view port 116 is shown. Also shown in FIG. 6A, are a cycle indicator 117 and an activation switch 107. The one or more covers do not necessarily need to slide. FIG. 6D is an isometric drawing of another embodiment of the sanitizing device described herein, showing the location of an entrance/exit port 106 for an electric cord on the left side of the unit. FIG. 6E is a close-up view of FIG. 6D.

The device described herein produces UV radiation, for example, near 254 nm, which is known to cause germicidal effects by interacting with the DNA of microorganisms and creating thymine dimers within the DNA strands causing the exposed microorganisms to be unable to function and reproduce. The low pressure mercury UV germicidal lamps employed in the device produce copious amounts of UV radiation from the strong mercury atomic radiation line centered at 253.7 nm, which is in the wavelength region between 220 nm and 300 nm where a broad peak occurs in germicidal effects as shown in FIG. 7.

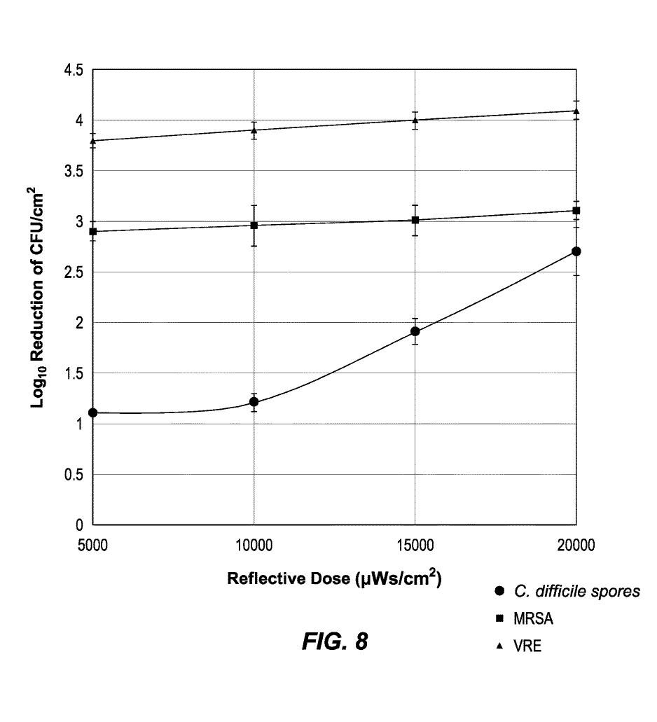

FIG. 8 shows data for UV destruction of three common pathogenic HAI organisms: C. diff, Methicillin-resistant Staphylococcus aureus and VRE. The data shows that a dose of 20,000 μW-s/cm² (20 mJ/cm²) reduces C. diff by approximately 2.7 logs, MRSA by about 3.1 logs, and VRE by about 4.1 logs. These are major reductions that would, significantly reduce the transmission of these pathogens from objects commonly handled by, for example, healthcare workers and patients.

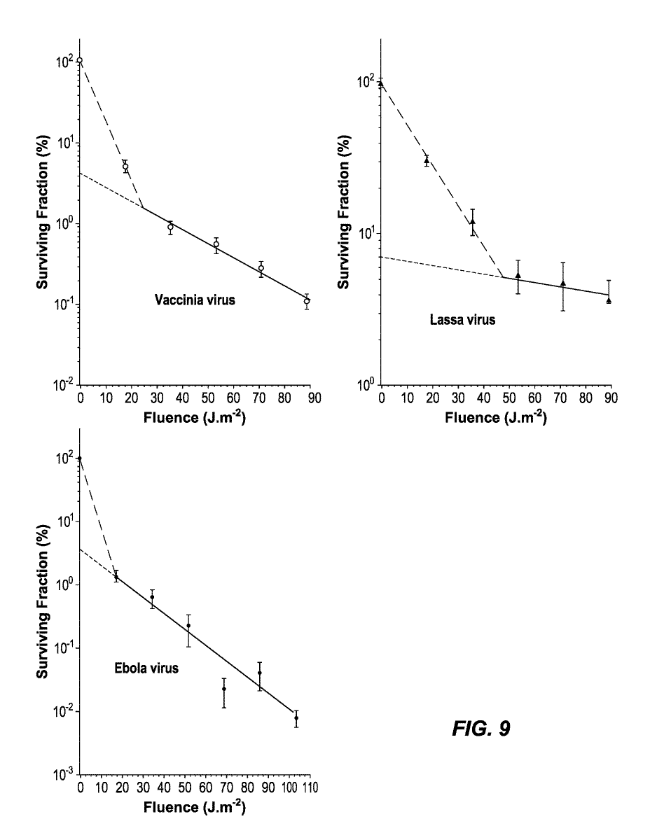

FIG. 9 shows data for destruction of three viruses associated with high fatality rates and person-to-person transmission. The data shown is for kill of: (A) vaccinia, a surrogate for the variola (smallpox) virus; (B) the Lassa virus that causes Lassa fever, a widespread disease in West Africa that affects up to 2 million people per year and results in 5,000 to 10,000 deaths per year, and (C) the Ebola virus that results in very high mortality rates for infected persons. The data shows that doses of approximately 100 J/m2 (10 mJ/cm²) of UV germicidal energy provides high kill levels for all three of these viruses. This data along with the data from FIG. 8 indicate that the application of more than about 10 mJ/cm² of UV energy to objects will significantly reduce the transmission of these major pathogens in healthcare facilities.

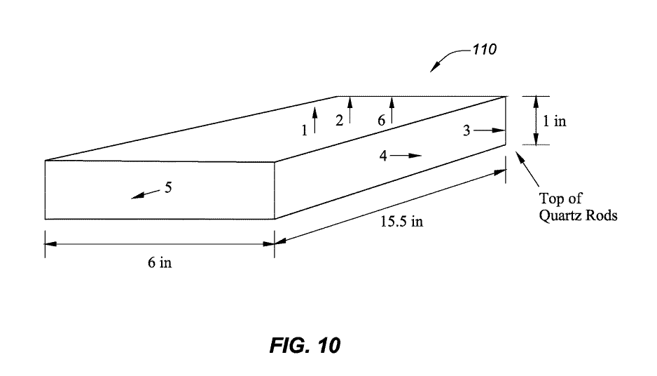

FIG. 10 shows an isometric drawing showing the location of test points for UV irradiance measurements in the UV sanitizer reflective chamber, as described below in the Examples section. Test Points: Test Point 1 (TP1): Center; Test Point 2 (TP2): Top, UV detector oriented up, 3.5 inches from longitudinal center; Test Point 3 (TP3): Side, UV detector oriented horizontally, 3.5 inches from longitudinal center, 0.5 inches below top; Test Point 4 (TP4): Side, UV detector oriented horizontally, in longitudinal center plane, 0.5 inches below top; Test Point 5 (TP5): End, UV detector oriented horizontally, in center; and Test Point 6 (TP6): Top, UV detector oriented up, 3.5 inches from longitudinal center, 1 inch from front surface of the chamber.

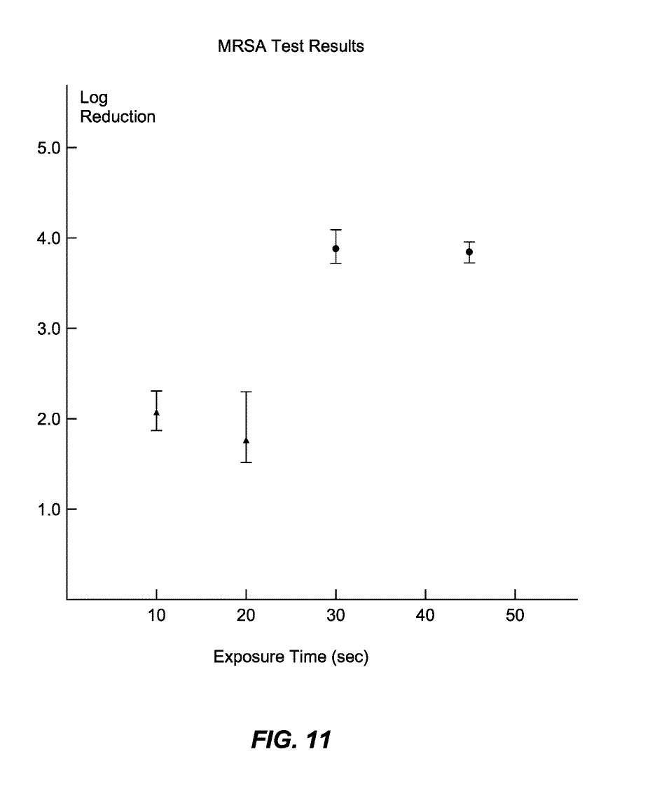

FIG. 11 shows data taken by an independent FDA certified test laboratory for kill of MRSA using a disinfection unit of the type described herein. MRSA organisms were inoculated onto metal coupons. Some of the coupons were then exposed to treatment in the disinfection unit and others were maintained as controls which were not exposed to treatment. Comparison of treated and untreated coupons showed that treatment in the disinfection unit produced approximately 4 logs (10,000 times) reduction in MRSA organisms on the coupons.

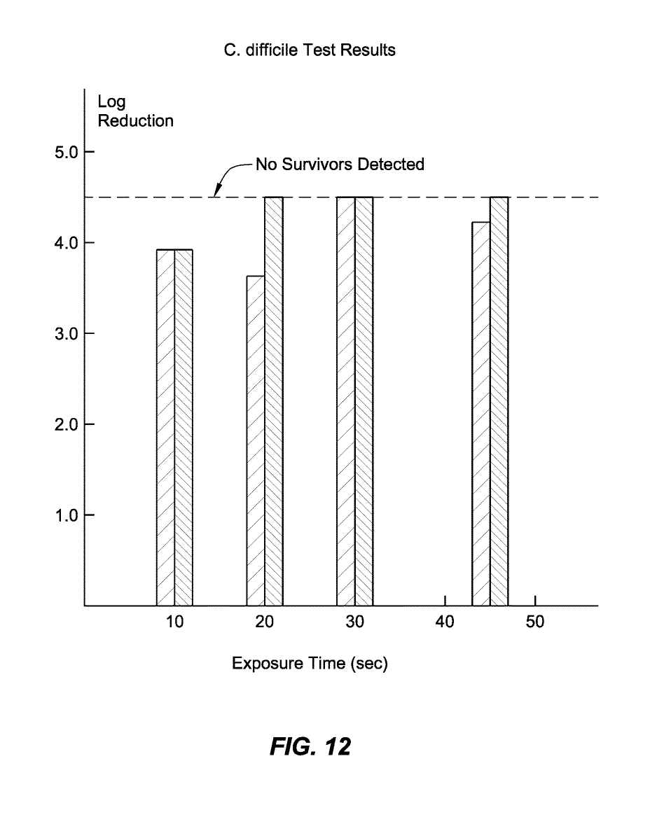

FIG. 12 shows data taken by an independent FDA certified test laboratory for kill of Clostridium difficile using a disinfection unit of the type described herein. The Clostridium difficile organisms were inoculated onto metal coupons. Some of the coupons were exposed to treatment in the disinfection unit and others were maintained as controls which were not exposed. Comparison of treated and untreated coupons showed that treatment in the disinfection unit produced approximately 4.5 logs (approximately 32,000 times) reduction in Clostridium difficile organisms.

### EXAMPLES

The following examples are intended to provide illustrations of the application of the present disclosure. The following examples are not intended to completely define or otherwise limit the scope of the disclosure. One of skill in the art will appreciate that many other methods known in the art may be substituted in lieu of the ones specifically described or referenced herein.

### Example 1: Testing of Internal UV Exposure Chambers

The device that was tested had internal chamber dimensions of 15.5 inches wide×9 inches high×6 inches deep. Tests were performed with three different internal UV exposure chambers: A) an internal UV exposure chamber fabricated with bare 5052 aluminum, B) an internal UV exposure chamber fabricated with a bare 5052 aluminum chamber painted with the UV-Max reflective paint described herein, and C) an internal UV exposure chamber fabricated with the Alanod Miro® 4300 UP UV reflective material described herein. The Alanod chamber (C) provided an increase in UV irradiance of approximately 1.9 times that of the bare aluminum chamber (A) and an increase in UV irradiance of approximately 1.8 times that of the UV-Max painted chamber (B). The uniformity of irradiance on an object 110 for each chamber was also measured using six different Test Points (see FIG. 10). The Alanod chamber showed improved uniformity compared to that of the other two chambers.

The UV dose measured in the center of the Alanod device with the UV detector oriented to look toward the top of the chamber (Test Point 1 as shown in FIG. 10) was approximately 220 mW-s/cm² for an exposure time of 45 seconds. Measurements at other locations (other Test Points) in the Alanod chamber found the minimum irradiation point to be near the end of the chamber with the UV detector oriented horizontally toward the end of the chamber (Test Point 5). The minimum irradiance was measured at Test Point 5 and was 35% of the irradiance measured at Test Point 1. The dose at the minimum irradiance point (Test Point 5) was approximately 77 mW-s/cm² for a 45 second exposure time.

### Example 2: Reduction of HAIs

UV measurements using a device, as shown in FIG. 1, show that the device can provide UV doses that are appropriate for achieving up to 4 logs reduction of microorganisms responsible for many HAIs in exposure times of less than 1 minute. The device that was tested had internal dimensions of 15.5 inches wide×9 inches high×6 inches deep.

Measurements of microbial kill were made in the device fabricated with the Alanod reflective material (as described in EXAMPLE 1). The microbiology tests were performed by an independent FDA certified test laboratory. These measurements showed approximately 4 logs kill of MRSA and approximately 4.5 logs kill of C. difficile as shown in FIG. 11 and FIG. 12. MRSA and C. difficile microorganisms were inoculated onto stainless steel coupons and placed in the center of the chamber for a 45 second exposure time. Controls were inoculated onto stainless steel coupons, but not exposed to the UV. Microorganisms from both the controls and the exposed samples were recovered and cultured using standard microbiology techniques and surviving colony forming units (CFUs) were enumerated. The kill levels were determined by comparing the number of CFUs in samples exposed to the UV treatment as compared to the controls.

The measured reduction levels of approximately 4 logs (10,000 times reduction) to approximately 4.5 logs (approximately 32,000 times reduction) would result in a major decrease in the likelihood of nosocomial infections occurring as the result of such microorganisms being transmitted from objects such as handheld devices in hospital or other healthcare environments.

While certain embodiments have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A chamber for sanitizing an object or the surface of an object, comprising:
a) an interior and an exterior;
b) one or more internal UV reflective surfaces comprising a UV reflective material;
c) one or more supports to place the object upon;
d) one or more light sources that emit ultraviolet light; and
e) a door on one surface of the chamber in which the object can enter or exit the chamber.

2. The chamber of claim 1, wherein the chamber further comprises:
a) a port connecting the interior of the chamber with the exterior of the chamber;
b) an electrical system;
c) an interlock switch;
e) an activation switch;
f) a cycle indicator; or
g) a view port indicator.

3. The chamber of claim 1, wherein
a) the UV reflective surface comprises a UV reflective material having a reflectivity of 50% to 60%, 60% to 70%, 70% to 80%, 80% to 90%, or 90% to 99.999%, or is coated with a UV reflective material having a UV reflectivity of 50% to 60%, 60% to 70%, 70% to 80%, 80% to 90%, or 90% to 99.999%;
b) the reflective material comprises: aluminum; Miro® 4300 UP UV; Polytetrafluoroethylene (PTFE); expanded PTFE (ePTFE), or a combination of aluminum, Miro® 4300 UP UV, Polytetrafluoroethylene (PTFE), or expanded PTFE (ePTFE);
c) the reflective material has a UV reflectivity of greater than 80%, or a UV reflectivity of 80% to 81%, 81% to 82%, 82% to 83%, 83% to 84%, 84% to 85%, 85% to 86%, 86% to 87%, 87% to 88%, 88% to 89%, 89% to 90%, 90% to 91%, 91% to 92%, 92% to 93%, 93% to 94%, 94% to 95%, 95% to 96%, 96% to 97%, 97% to 98%, 98% to 99%, or 99% to 100%; or
d) the reflective material is a surface coated with a UV reflective paint.

4. The chamber of claim 1, wherein
a) the one or more supports comprise quartz; or
b) the one or more supports comprise a pocket in which the object can be placed.

5. The chamber of claim 1, wherein the emitted ultraviolet light is from about 100 nm to about 300 nm, from about 220 nm to about 280 nm, from about 240 nm to about 280 nm, from about 220 nm to about 320 nm, or from about 250 to 270 nm, or about 254 nm or about 265 nm.

6. A method of sanitizing an object or the surface of an object, comprising:
a) obtaining a chamber of claim 1;
b) placing the object through the door of the chamber;
c) placing the object onto the one or more supports;
d) closing the door; and
e) turning on the one or more light sources emitting ultraviolet light.

7. A method of reducing microbes on a surface of an object, comprising:
a) obtaining a chamber of claim 1;
b) placing the object through the door of the chamber;
c) placing the object onto the one or more supports;
d) closing the door;
e) turning on the one or more light sources emitting ultraviolet light; and
f) reducing the microbes on the surface about 1 to about 5 logs magnitude or about 0.5 to about 5 logs magnitude.

8. A chamber for sanitizing an object or the surface of an object, comprising:
a) an interior chamber comprising one or more internal UV reflective surfaces comprising a UV reflective material;
b) an outer chamber comprising an exterior surface;
c) one or more supports to place the object upon;
d) one or more light sources that emit ultraviolet light; and
e) a door on one surface of the chamber in which the object can enter or exit the chamber.

9. The chamber of claim 8, wherein the chamber further comprises:
a) a port connecting the exterior surface of the outer chamber with the internal UV reflective surfaces of the interior chamber;
b) an electrical system;
c) an interlock switch;
e) an activation switch;
f) a cycle indicator; or
g) a view port indicator.

10. The chamber of claim 8, wherein
a) the reflective surface comprises a UV reflective material having a UV reflectivity of 50% to 60%, 60% to 70%, 70% to 80%, 80% to 90%, or 90% to 99.999%, or is coated with a UV reflective material having a UV reflectivity of 50% to 60%, 60% to 70%, 70% to 80%, 80% to 90%, or 90% to 99.999%;
b) the reflective material comprises: aluminum; Miro® 4300 UP UV; Polytetrafluoroethylene (PTFE); expanded PTFE (ePTFE), or a combination of aluminum, Miro® 4300 UP UV, Polytetrafluoroethylene (PTFE), or expanded PTFE (ePTFE);
c) the reflective material has a UV reflectivity of greater than 80%, or a UV reflectivity of 80% to 81%, 81% to 82%, 82% to 83%, 83% to 84%, 84% to 85%, 85% to 86%, 86% to 87%, 87% to 88%, 88% to 89%, 89% to 90%, 90% to 91%, 91% to 92%, 92% to 93%, 93% to 94%, 94% to 95%, 95% to 96%, 96% to 97%, 97% to 98%, 98% to 99%, or 99% to 100%; or
d) the reflective material is a surface coated with a UV reflective paint.

11. The chamber of claim 8, wherein
a) the one or more supports comprise quartz; or
b) the one or more supports comprise a pocket in which the object can be placed.

12. The chamber of claim 8, wherein the emitted ultraviolet light is from about 100 nm to about 300 nm, from about 220 nm to about 280 nm, from about 240 nm to about 280 nm, from about 220 nm to about 320 nm, or from about 250 to 270 nm, or about 254 nm or about 265 nm.

13. A method of sanitizing an object or the surface of an object, comprising:
a) obtaining a chamber of claim 8;
b) placing the object through the door of the chamber;
c) placing the object onto the one or more supports;
d) closing the door; and
e) turning on the one or more light sources emitting ultraviolet light.

14. A method of reducing microbes on a surface of an object, comprising:
a) obtaining a chamber of claim 8;
b) placing the object through the door of the chamber;
c) placing the object onto the one or more supports;
d) closing the door;
e) turning on the one or more light sources emitting ultraviolet light; and
f) reducing the microbes on the surface about 1 to about 5 logs magnitude or about 0.5 to about 5 logs magnitude.
